(19) United States
(12) Patent Application Publication
(10) Pub. No.: US 2004/0215276 A1
(43) Pub. Date: Oct. 28, 2004

(54) Form analysis to detect evoked response

(76) Inventors: Patrick Scholten, Zutphen (NL); Peter Oosterhoff, Zutphen (NL); Henricus W.M. De Bruyn, Arnhem (NL); Geeske Van Oort, Velp (NL)

Correspondence Address: MEDTRONIC, INC., 710 MEDTRONIC PARKWAY NE, MS-LC340, MINNEAPOLIS, MN, 55432-5604, US

(21) Appl. No.: 10/424,538

(22) Filed: Apr. 25, 2003

(51) Int. Cl. A61N 1/37

(52) U.S. Cl. 607/28

(57) ABSTRACT

In general, the invention is directed to techniques for determining capture status of a heart chamber that receives a pulse from an implantable pulse generator (IPG). Digital signal processing (DSP) can be used to improve the reliability of capture detection by transforming the sensed response signal into a set of morphological characteristics. Analysis of selected morphological characteristics serves to distinguish signals indicative of capture from signals indicative of loss of capture.

### FIELD OF THE INVENTION

The invention relates to implantable medical devices having an implantable pulse generator (IPG) for cardiac stimulation, and more particularly, to detection of evoked response for capture management.

### BACKGROUND OF THE INVENTION

An implantable medical device such as a cardiac pacemaker supplants some or all of the natural pacing function of a heart by delivering appropriately timed electrical stimulation signals designed to cause the myocardium of the heart to contract. An implantable pulse generator (IPG) in the device generates the electrical stimuli. To be effective, the stimuli should be of a sufficient strength (or amplitude) and duration (or pulse width) to cause the heart to beat, i.e., to “capture” the heart. A “capture threshold” or “stimulation threshold” defined by a strength-duration curve separates stimuli that capture the heart from stimuli that fail to capture the heart.

Because a failure to capture the heart may result in serious complications or death, pacing stimuli are generally delivered with a strength and duration above the capture threshold by a safety margin. It is generally desirable, however, that the safety margin be reasonably large enough to ensure capture but small enough that power not be wasted. Implantable medical devices that draw power from a battery have a limited power supply, and the strength and duration of the stimuli should be regulated to prolong battery life. The strength and duration of stimuli may be set or adjusted with programming equipment that communicates with the implantable medical device.

There are various means of determining whether a capture occurred. A common method for determining capture success involves sensing a response signal from the heart and comparing the signal to a capture threshold. A signal with amplitude greater than the capture threshold is interpreted as an evoked response indicative of capture or “CAP.” A signal with an amplitude less than the capture threshold indicates loss of capture or “LOC.” U.S. Pat. No. 6,067,472 to Vonk et al. describes an existing technique for evoked response detection.

### SUMMARY OF THE INVENTION

In general, the invention is directed to techniques for determining capture status of a heart chamber that receives a pulse from an implantable pulse generator (IPG). The capture status indicates that the pulse resulted in CAP or LOC for a chamber of the heart to which a pacing pulse is applied. Successful capture results in an evoked response from the heart.

It is often very difficult to distinguish an evoked response indicating CAP from post-pace signals present in the case of LOC. For example, unstable polarization can distort the CAP and LOC signals such that their amplitudes are very similar. The distortion may lead to inaccurate detection of heart capture. In accordance with the invention, a digital signal processor (DSP) is used to improve the reliability of capture detection by transforming the sensed response signal into a set of morphological characteristics.

Analysis of selected morphological characteristics serves to distinguish signals indicative of CAP from signals indicative of LOC. The invention uses analysis of one or more morphology characteristics with or without amplitude-threshold analysis to make a distinction between CAP and LOC. The signal morphology characteristic may include at least one of: a maximum slope of the sensed signal, a time of maximum slope of the sensed signal, a minimum slope of the sensed signal, a width of the signal, a minimum voltage of the sensed signal, a maximum voltage of the signal, a time of minimum voltage, a time of maximum voltage, and a minimum slope of the sensed signal. Each of the above morphology characteristics differs sufficiently between CAP and LOC so as to serve as a basis for the CAP versus LOC determination.

The use of multiple morphology characteristics in determining the capture status gives more reliable results than the use of just one morphology characteristic. Accordingly, the invention further contemplates the correlation of multiple morphology characteristics to enhance selectivity in classifying cardiac signals as indicating CAP or LOC.

In one embodiment, the invention is directed to a method comprising delivering a pacing pulse to a chamber of the heart, sensing a signal within the chamber following the delivery of the pacing pulse, and determining whether the pacing pulse captured the chamber of the heart based on one or more morphological characteristics of the sensed signal.

In another embodiment, an implantable medical device comprises a sensor to sense a signal from within a chamber of a heart following delivery of a pacing pulse, and a processor to determine whether the pacing pulse captured the chamber of the heart based on one or more morphological characteristics of the sensed signal. Additionally, the implantable medical device may comprise a lead having a proximal end and a distal end, the lead comprising an electrode on the distal end. The implantable medical device may also comprise a pulse generator to generate a pacing pulse for delivery to a chamber of the heart via the electrode.

The invention further includes computer-readable media comprising instructions for causing a programmable processor to carry out the methods described above.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic view of an exemplary implantable medical device implanted within a human body.

FIG. 2 is a diagram of the implantable medical device of FIG. 1 located in and near a heart.

FIG. 3 is a block diagram illustrating the constituent components of the implantable medical device depicted in FIGS. 1 and 2.

FIG. 4 is a flow diagram illustrating a technique for determining capture status.

FIG. 5 is a flow diagram illustrating the technique of FIG. 4 in greater detail.

FIG. 6 is a histogram illustrating a minimum voltage of a rough sensed signal.

FIG. 7 is a histogram illustrating a minimum voltage of a filtered signal.

FIG. 8 is a histogram illustrating a maximum slope of a filtered signal.

FIG. 9 is a histogram illustrating a time of maximum slope or a filtered signal.

FIG. 10 is a histogram illustrating a minimum slope of a filtered signal.

FIG. 11 is a histogram illustrating a period between first and last threshold crossing.

### DETAILED DESCRIPTION OF THE INVENTION

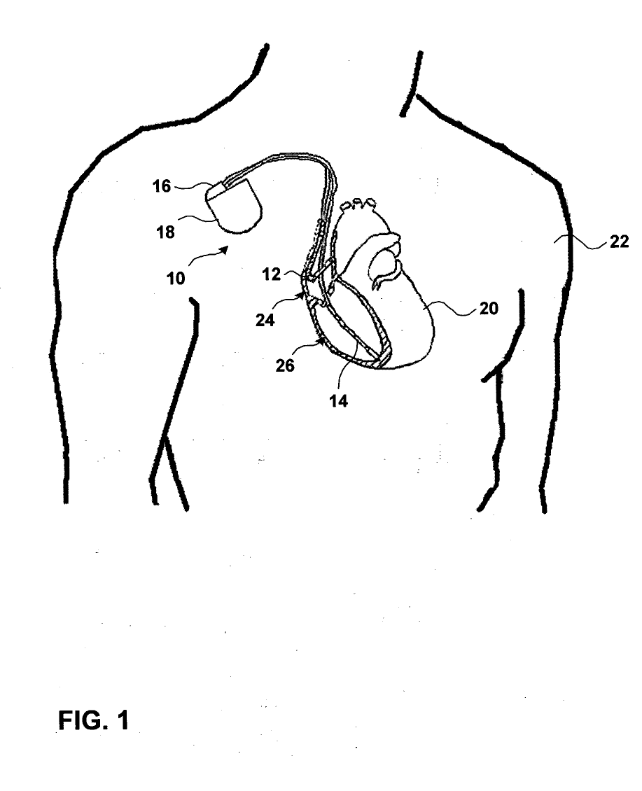

FIG. 1 is a schematic view of an exemplary implantable medical device (IMD) 10 implanted within a human patient 22. IMD 10 is an implantable pacemaker that may include cardioversion and defibrillation capability. The invention is not limited to the particular IMD shown in FIG. 1, however, but may be practiced by any number of implantable devices. The techniques of the invention may be practiced by a device that paces a single cardiac chamber or several chambers, that paces one or more atria or one or more ventricles, that includes or lacks cardioversion and defibrillation capability, and that paces in any pacing mode.

The invention is directed to techniques for determining capture status of a heart chamber that receives a pacing pulse from an implantable pulse generator incorporated in IMD 10. The capture status indicates whether the pacing pulse successfully captured the chamber of the heart to which the pacing pulse was applied. Accordingly, capture status may indicate capture (CAP) or loss of capture (LOC). Successful capture results in an evoked response from the heart. It is often very difficult to distinguish an evoked response indicating CAP from post-pace signals present in the case of LOC. For example, unstable polarization can distort the CAP and LOC signals such that their amplitudes are very similar. The distortion may lead to inaccurate detection of heart capture.

In accordance with the invention, IMD 10 improves the reliability of capture detection by generating a set of morphological characteristics from the sensed signal. IMD 10 may employ, for example, digital signal processing (DSP) to identify the morphological characteristics. Analysis of selected morphological characteristics serves to distinguish signals indicative of CAP from signals indicative of LOC. For purposes of illustration, this description refers extensively to several morphology characteristics that may be used in determining capture status. These are simply example characteristics, and the invention is not necessarily limited to the morphology characteristics described herein.

In the example of FIG. 1, IMD 10 is a pacemaker comprising atrial pacing and sensing lead 12 and ventricular pacing and sensing lead 14 attached to connector module 16 of hermetically sealed enclosure 18 and implanted near human or mammalian heart 20 of patient 22. Pacing and sensing leads 12 and 14 sense electrical signals attendant to the depolarization and depolarization of the heart 20, and further provide pacing pulses for causing depolarization of cardiac tissue in the vicinity of the distal ends thereof. Leads 12 and 14 may have unipolar or bipolar electrodes disposed thereon. IMD 10 is one example of a device capable of practicing the invention, in that IMD 10 has the capability of sensing a signal from within a chamber of a heart following delivery of a pacing pulse, and determining whether the pacing pulse captured the chamber of the heart based on one or more morphological characteristics of the sensed signal. The signal may be an entire sensed signal within an analysis window or only a portion of an evoked response.

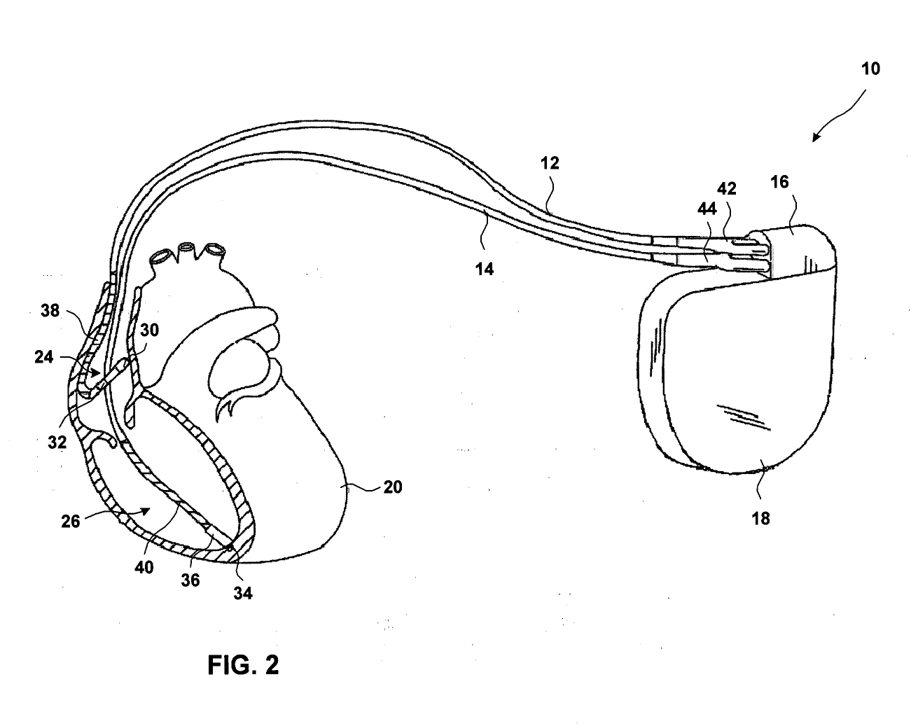

FIG. 2 is a diagram of IMD 10 of FIG. 1 located in and near heart 20. FIG. 2 shows IMD 10 with connector module 16 and hermetically sealed enclosure 18. Atrial and ventricular pacing leads 12 and 14 extend from connector module 16 to the right atrium 24 and right ventricle 26, respectively, of heart 20. In some embodiments, IMD 10 also may include left atrial and/or ventricular leads. Atrial electrodes 30 and 32 disposed at the distal end of atrial pacing lead 12 are located in right atrium 24. Ventricular electrodes 34 and 36 disposed at the distal end of ventricular pacing lead 14 are located in right ventricle 26.

A pulse generator (not shown in FIG. 2) inside enclosure 18 generates pacing pulses. The pacing pulses are delivered to right atrium 24 or right ventricle 26 by electrodes 30, 32, 34, 36. In accordance with the invention, IMD 10 comprises a sensor to sense a signal from within a chamber of heart 20 following delivery of a pacing pulse, and a processor (not shown in FIG. 2) to determine whether the pacing pulse captured the chamber of heart 20 based on one or more morphological characteristics of the sensed signal. IMD 10 may further comprise a lead having a proximal end and a distal end, the lead comprising an electrode on the distal end. IMD 10 may also comprise a pulse generator to generate a pacing pulse for delivery to a chamber of the heart via the electrode.

In addition to pacing, IMD 10 may apply other forms of therapy. In FIG. 2, for example, atrial lead 12 and ventricular lead 14 include defibrillation electrodes 38 and 40, respectively. Defibrillation electrodes 38 and 40 deliver defibrillation shocks to right atrium 24 or right ventricle 26 when necessary to terminate an episode of atrial or ventricular fibrillation. Atrial and ventricular leads 12, 14 each include an elongated insulative lead body carrying one or more conductors insulatively separated from one another. At the proximal ends of leads 12, 14 are bifurcated connectors 42, 44, which electrically couple the connectors to connector module 16 of IMD 10.

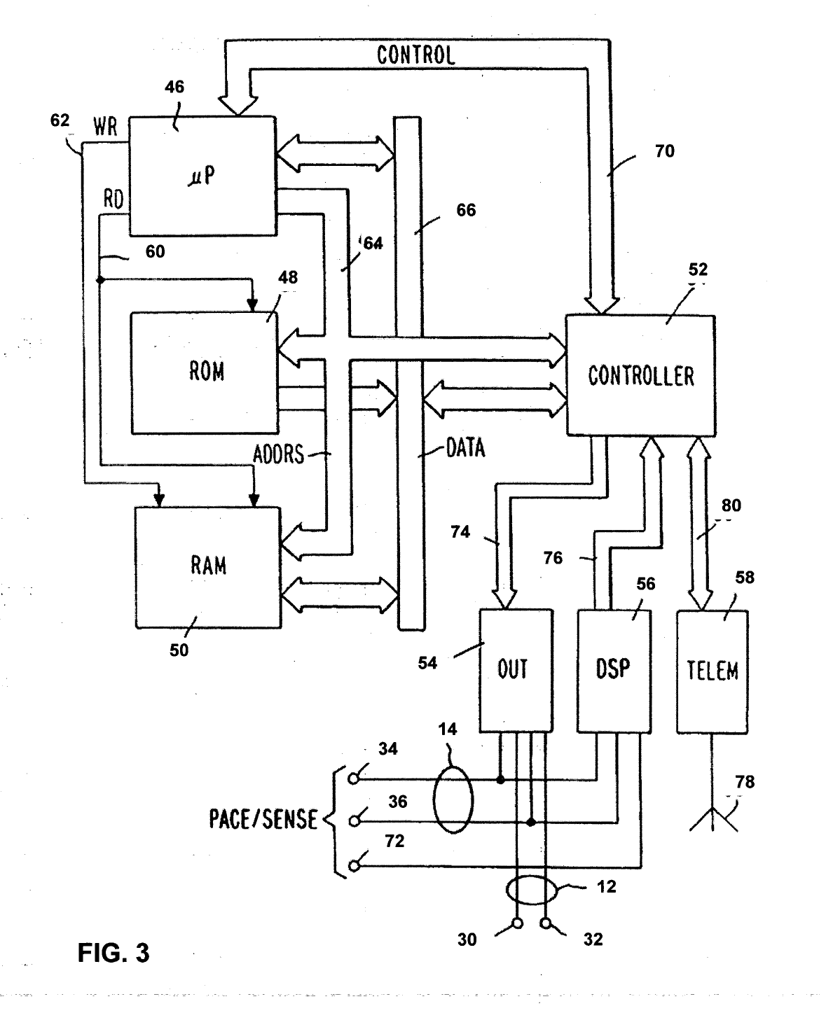

FIG. 3 shows a block diagram illustrating exemplary components of IMD 10 in accordance with one embodiment of the invention, in which IMD 10 is a pacemaker having a microprocessor-based architecture. The components depicted in FIG. 3 may be arranged and operate substantially as described in commonly assigned U.S. Pat. No. 6,029,087 to Wohlgemuth, the entire content of which is incorporated herein by reference. It is to be noted that FIG. 3 is representative of IMD 10, and is not limiting in the actual architecture of the pacemaker. It is presented for the purpose of discussing data flow and, in particular, the position of a DSP chip and a microprocessor for purposes of sensing, analyzing and classifying sensed intracardiac signals. Accordingly, FIG. 3 is considered to be exemplary rather than limiting with regard to the present invention. While the invention is disclosed as embodied in a pacemaker, it is likewise applicable to incorporation in a cardioverter, or combined cardioverter pacemaker, cardioverter defibrillator pacemaker, and the like. Further, while the discussion of FIG. 3 assumes a single chamber ventricular pacing system for purposes of illustration, it is to be understood that the invention is applicable to dual chamber, as in FIGS. 1 and 2, and multi-chamber systems. For example, in a dual chamber embodiment, the DSP chip may have three channels, for respective processing of P, R and T wave signals.

The primary elements of the exemplary IMD 10 illustrated in FIG. 3 are microprocessor 46, read only memory 48, random access memory 50, a digital controller 52, output amplifier 54, DSP circuitry 56, and a telemetry/programming unit 58. Read only memory 48 stores the basic programming for the device, including the primary instructions set defining the computations performed to derive the various timing intervals performed by the device. Random access memory 50 serves to store the values of variable control parameters, such as programmed pacing rate, pulse widths, pulse amplitudes, and so forth, which are programmed into the device by the physician. Reading from random access memory 48 and read only memory 50 is controlled by RD-line 60. Writing to random access memory 50 is controlled by WR-Line 62. In response to a signal on RD-Line 60, the contents of random access memory 50 or read only memory 48 designated by the then present information on address bus 64 are placed on data bus 66. Similarly, in response to a signal on WR-line 62, information on data bus 66 is written into random access memory 50 at the address specified by the information on address bus 64.

Controller 52 performs all of the basic timing and control functions of the illustrative pacemaker device. Controller 52 includes at least one programmable timing counter, e.g., initiated on paced or sensed ventricular contractions, for timing out intervals thereafter. This timing counter is used to define the escape intervals for timing generation of pace pulses, as well as for timing the respective durations of the charge and recharge pulse portions of triphasic pulses. Controller 52 triggers output pulses to be generated and delivered from output stage 54, and it generates interrupts on control bus 70 for cyclically waking microprocessor 46 from its sleep state to allow it to perform the required functions. For a single chamber pacemaker, output stage 54 is coupled to electrodes 34 and 36 which are employed both for delivery of pacing pulses and for sensing of cardiac signals. Electrode 36 is typically located on the distal tip end of endocardial ventricular lead 14, and for ventricular pacing is preferably placed in the apex of the right ventricle. Electrode 34 is preferably a ring electrode, as used with a bipolar lead. Electrode 72 represents the IMD housing 18, or “can,” which may be used as the indifferent electrode for selected unipolar pacing and/or sensing operations. Of course, for a dual or multi-chamber pacing system, additional electrodes are employed. For example, electrodes 30, 32 carried by lead 12 may be used for pacing and sensing in the atrium, while electrodes 34, 36 are used in the ventricle. Output circuit 54 is controlled by controller 52 through bus 74 to determine the amplitude and pulse width of the pulse to be delivered and to determine which electrode pair is to be employed to deliver the pulse.

Cardiac signals are sensed at a desired pair or pairs of electrodes; bipolar and/or unipolar sensing may be used. For “combipolar” sensing, a unipolar lead in the atrium and a unipolar lead in the ventricle are used, e.g., the signals are picked up at electrodes 30, 34. Sense signals are inputted to DSP 56, which comprises a number of signal processing channels corresponding to signals of interest. For example, in a dual chamber pacemaker which incorporates P wave processing either for rate control, capture detection or any other reason, there are three channels for respective signal processing of the P, R and T waves. The data resulting from the digital signal processing is transmitted via bus 76 through controller 52 and bus 70 to microprocessor 46, for signal classification operations, as well as any other necessary calculations.

External control of the implanted device is accomplished via telemetry/control block 58, which allows communication between the implanted device and an external programmer (not shown). Radio communication is typically employed via antenna 78. Appropriate telemetry/programming systems are well known in the art. The present invention is workable with any conventional telemetry/programming circuitry. Information entering the pacemaker from the programmer is passed to controller 52 via bus 80. Similarly, information from the pacemaker is provided to the telemetry block 58 via bus 80, for transmission to the external programmer. The classification algorithms for processing the parameters generated by each DSP channel can be re-programmed in a known manner, as described in the above-referenced Wohlgemuth patent. In particular, IMD 10 carries instructions that cause processor 46 to determine whether a pacing pulse captured a chamber of heart 20 based on one or more morphological characteristics of a sensed signal. The morphological characteristics are determined by DSP 56. For example, the above-referenced Wohlgemuth patent describes in detail how different morphology parameters can be determined from different signals. Accordingly, the invention further contemplates computer-readable media comprising instructions for execution by processor 46 and/or DSP to process and analyze the morphological characteristics.

DSP 56 digitally processes the sensed signal to identify the morphology characteristic. Processor 46 compares the identified morphology characteristic to one or more morphology criteria, and determines that the pacing pulse captured the chamber when the morphology characteristics satisfy the morphology criteria. Additionally, processor 46 determines that the pacing pulse did not capture the chamber if the morphology characteristics do not satisfy the morphology criteria.

As discussed above, processor 46 and DSP 56 work together to apply digital signal processing and analysis techniques to characterize the digitized signals stored in RAM 50, or received in real time, to recognize and classify the patient's heart rhythm or to analyze the morphology of the signals employing any of several signal processing methodologies. During digital signal analysis, various cardiac parameters may be measured, such as the S-T segment, i.e., the duration between the conclusion of the depolarization marked by the QRS complex and the onset of repolarization marked by the T-wave. In this manner, processor 46 performs a capture test to determine whether there is a possibility of LOC.

In accordance with the invention, processor 46 analyzes one or more morphology characteristics that are indicative of a potential for LOC. In particular, the signal morphology characteristics may include at least one of: a maximum slope of the sensed signal, a time of maximum slope of the sensed signal, a minimum slope of the sensed signal, a width of the signal, a minimum voltage of the sensed signal, a maximum voltage of the signal, a time of minimum voltage, a time of maximum voltage, and a minimum slope of the sensed signal. In some embodiments, IMD 10 may execute a learning sequence in which the IMD evaluates various morphological characteristics, or combinations thereof, to identify those characteristics that are most useful in classifying CAP versus LOC.

The signal may be the rough sensed signal, or the additional filtered sensed signal, or the slope of the filtered sensed signal. The values of each of the above morphology characteristics serve to distinguish post-pacing responses that are indicative of an evoked response caused by CAP from those indicative of LOC. IMD 10 may rely on a single morphology characteristics or a correlation provided by multiple morphology characteristics to identify CAP or LOC. In other words, IMD 10 may rely on individual characteristics, combinations of characteristics, or complex morphological templates.

As one example, IMD 10 may use a minimum voltage of the sensed signal for a morphology characteristic. The minimum voltage represents the minimum voltage of the sensed signal within an applicable sampling window. The minimum voltage characteristic is compared with morphology criteria in the form of a minimum voltage threshold. If the minimum voltage of a sensed signal is within the range set by the range of the minimum voltage criteria, processor 46 determines that the pacing pulse captured heart 20.

IMD 10 senses a signal from within a chamber of heart 20 following delivery of a pacing pulse via a sense electrode deployed on a cardiac lead deployed within the chamber. A sense amplifier can be provided to amplify the sensed signal, which is converted to digital representation by an analog-to-digital converter. An analog and digital filter serves to filter the signal. DSP 56 processes the resulting digital signal, and processor 46 determines whether the pacing pulse captured the chamber of heart 20 based on one or more morphological characteristics of the sensed signal.

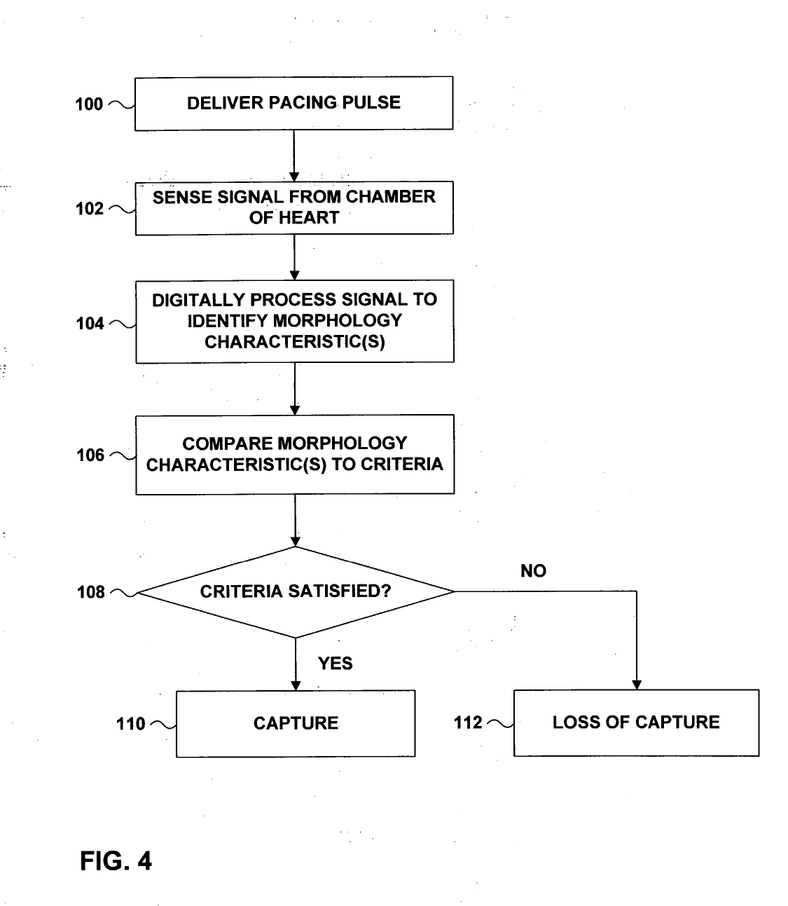

FIG. 4 is a flow diagram illustrating a technique for determining capture status. Upon delivery of a pacing pulse to a chamber of the heart (100), IMD 10 senses a signal within the chamber (102). In particular, IMD 10 senses whether the chamber emits an evoked response to the pacing pulse, indicating CAP. Again, the signal may include distortion that can lead to inaccurate capture detection. As shown in FIG. 4, a processor within IMD 10 digitally processes the sensed signal to identify one or more morphological characteristics that describe the behavior of the heart (104).

The signal morphology characteristics may include at least one of: a maximum slope of the sensed signal, a time of maximum slope of the sensed signal, a minimum slope of the sensed signal, a width of the signal, a minimum voltage of the sensed signal, a maximum voltage of the signal, a time of minimum voltage, a time of maximum voltage, and a minimum slope of the sensed signal. Each of the above morphology characteristics are particularly useful in distinguishing sensed signal waveforms indicating CAP from those indicating LOC. Additionally, other parameters may be used.

To further increase the accuracy of capture detection, threshold analysis, which compares the amplitude of a sensed signal to a predefined threshold, may be used along with the described morphology-based technique. A value below the threshold is considered to be indicative of LOC, while a value above the threshold is considered to be indicative of CAP. Satisfaction of the threshold, coupled with satisfaction of applicable morphology characteristics by one or more of the morphology characteristics, permits a more accurate CAP determination.

IMD 10 compares the identified morphology characteristics to criteria (106) that specify values for the applicable one or more morphology characteristics. Comparison of a single morphology characteristic, with or without a threshold comparison, may provide a sufficiently accurate way to evaluate CAP versus LOC. However, using a plurality of morphology characteristics in the comparison gives more accuracy and selectivity in determining capture status.

The criteria considered in the comparison defines a threshold value or range for each morphology characteristic such that a value that falls within the applicable range or above or below the applicable threshold value corresponds to CAP. if the morphology characteristic(s) satisfy the applicable morphology criteria (108), IMD 10 indicates that the chamber of the heart was successfully captured (110). On the other hand, if the criteria were not satisfied, the invention indicates a loss of capture (112).

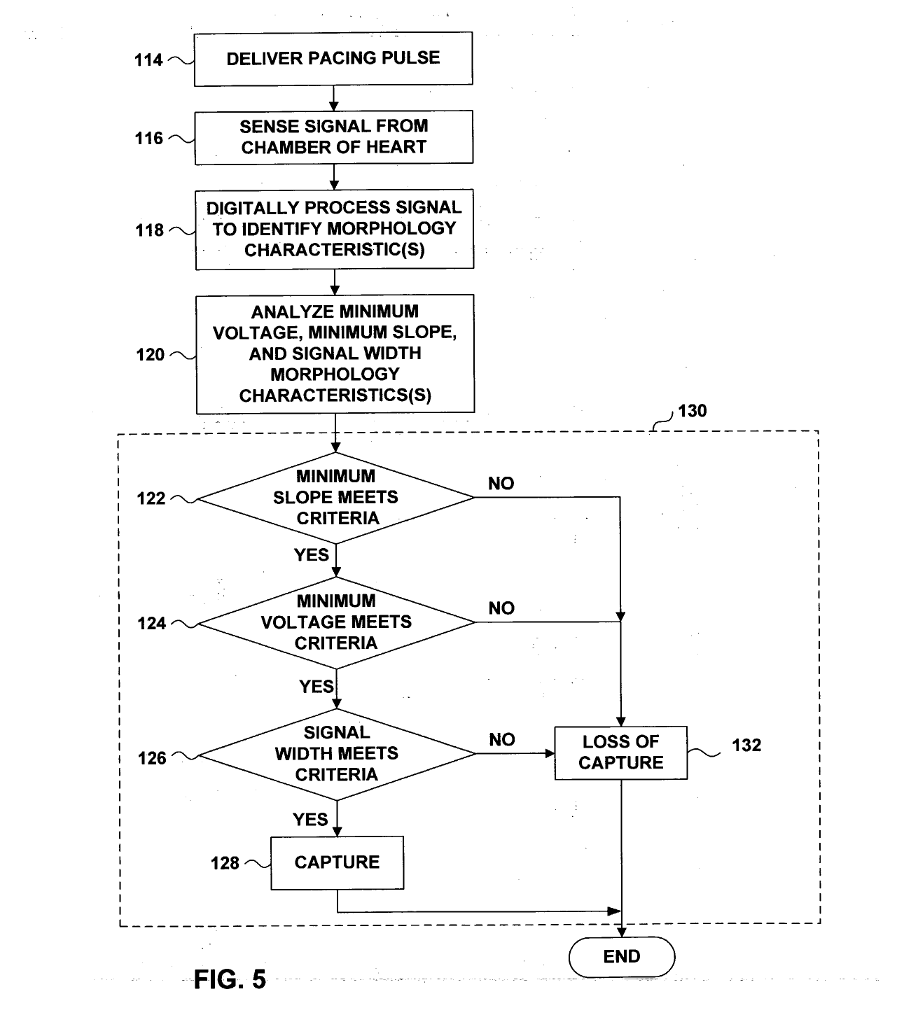

FIG. 5 is a flow diagram illustrating the technique of FIG. 4 in greater detail. Upon delivery of a pacing pulse to a chamber of the heart (114), IMD 10 senses a signal from the heart chamber (116), and identifies one or more morphology characteristics (118).

IMD 10 then analyzes the individual morphology characteristics relative to applicable morphology criteria (120). For example, in FIG. 5, IMD 10 compares minimum voltage, minimum slope, and signal width morphology characteristics of the sensed signal to pertinent criteria. These three morphology characteristics are described for purposes of illustration, and typically provide very high selectivity in distinguishing CAP from LOC, as will be discussed in greater detail. In general, the example of FIG. 5 represents the analysis of multiple morphology characteristics, rather than a single characteristics, in an effort to correlate the characteristics and provide a more accurate CAP versus LOC determination. In some embodiments, IMD 10 may use sophisticated classification algorithms to process multiple parameters as input characteristics for a classification engine.

The identified morphology characteristics are compared to criteria (121) that include three morphology characteristics in this example. Using a plurality of morphology characteristics in the comparison gives more accuracy in determining capture status. The criteria may define a range for each morphology characteristic such that a value within the range corresponds to a capture and a value outside the range corresponds to a loss of capture. In particular, the minimum slope of the sensed signal is compared to the minimum slope criteria (122).

If the minimum slope is within the range specified by the criteria, IMD 10 compares the minimum voltage of the sensed signal with the minimum voltage criteria. If the minimum voltage of the sensed signal is within the range specified by the minimum voltage criteria (124), the signal width of the sensed signal is compared with the signal width criteria. If the signal width of the sensed signal falls within the range specified by the signal width criteria (126), the processor determines that the pulse successfully captured the heart (128). If any of the morphology characteristics do not meet the corresponding criteria, however, IMD 10 determines that the pulse resulted in a loss of capture (132).

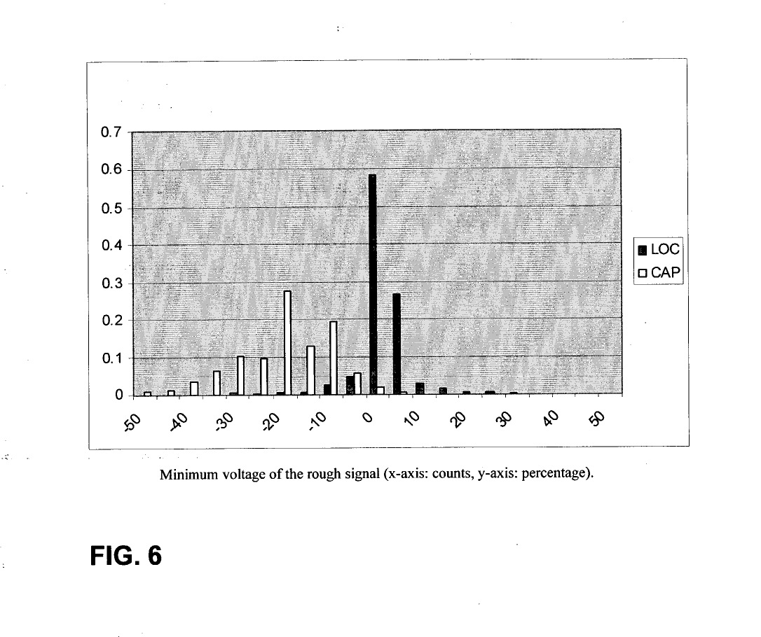

FIG. 6 is a histogram illustrating a minimum voltage of a rough signal sensed within a heart chamber within a sampling window following delivery of a pacing pulse. The “rough” signal is the sensed signal prior to significant processing or filtering. The minimum voltage characteristic is an example morphology characteristic that may be obtained from a sensed signal via digital signal processing. The characteristic may be used in determining the capture status of a heart. A processor converts the sensed analog voltage signals to digital values. The units of the digital values, as seen on the x-axis, are “counts,” which represent a signal level. The absolute range of the x-axis in the example of FIG. 6 is ±25 mV. The y-axis describes the occurrence-frequency, with 1 being equal to 100% occurrence rate. The data from the graphs or FIGS. 6-11 represent the results of an actual ventricular capture threshold test, involving thirty-four patients, and 3684 sensed beats.

When the minimum voltage morphology characteristic is greater than or equal to zero, the graph of FIG. 6 shows the probability of LOC is almost one. Therefore, an example criterion for this morphology characteristic may contain a threshold at zero, wherein the values below the threshold indicate a high probability of CAP. Conversely, a criterion for this morphology characteristic may contain a threshold at zero, wherein the values greater than or equal to zero indicate a low probability of CAP, i.e., a high probability of LOC. Accordingly, as evidenced by FIG. 6, the minimum voltage of a rough sensed signal may serve as a useful morphology characteristic to distinguish CAP versus LOC following delivery of a pacing pulse.

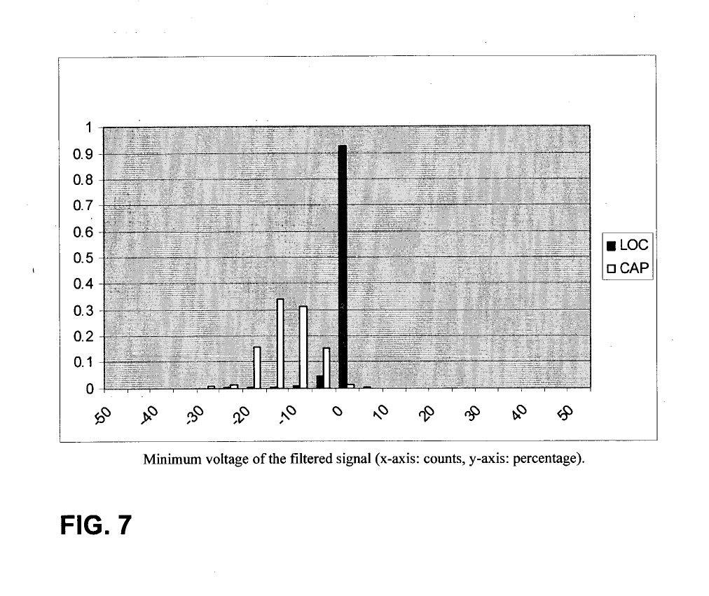

FIG. 7 is a histogram illustrating a minimum voltage of a filtered signal sensed within a heart chamber following delivery of a pacing pulse. The minimum voltage characteristic is an example of another morphology characteristic that may be obtained from a sensed signal. The units of the digital values, as seen on the x-axis, are “counts.” The absolute range of the x-axis in the example of FIG. 7 is ±50 mV. The y-axis describes the occurrence-frequency, with 1 being equal to 100% occurrence rate.

The graph of FIG. 7 shows that the filtered minimum voltage has an even more pronounced LOC range than the rough sensed minimum voltage characteristic. When the minimum voltage morphology characteristic is greater than or equal to zero, the probability of a loss of capture is almost one. Therefore, an example criterion for this morphology characteristic may contain a threshold at zero, wherein the values below the threshold indicate a high probability of capture. Conversely, a criterion for this morphology characteristic may contain a threshold at zero, wherein the values greater than zero indicate a high probability of a loss of capture.

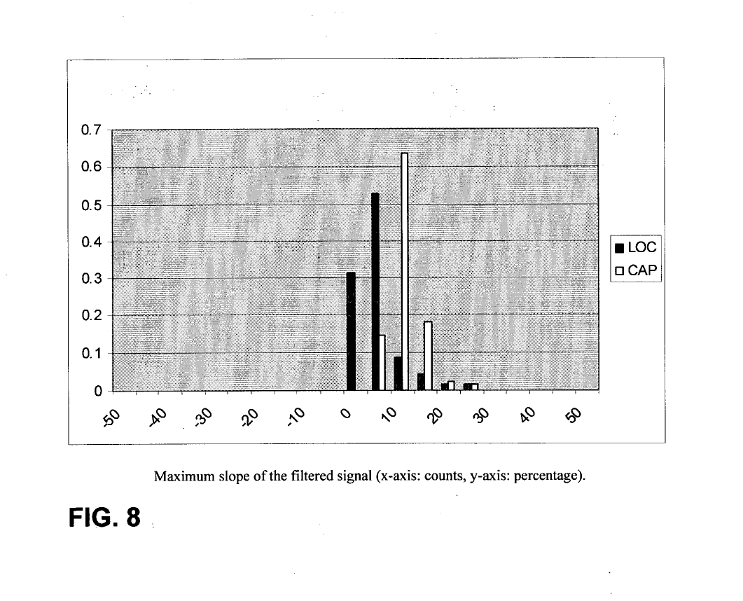

FIG. 8 is a histogram illustrating a maximum slope of a filtered signal sensed within a heart chamber following delivery of a pacing pulse. The maximum slope characteristic is another example morphology characteristic. The units of the digital values, as seen on the x-axis, are “counts.” The absolute range of the x-axis in the example of FIG. 8 is ±100 mV. The y-axis describes the occurrence-frequency, with 1 being equal to 100% occurrence rate.

When the maximum slope morphology characteristic is greater than or equal to approximately 10, the graph shows the probability of a loss of capture is almost one. Therefore, an example criterion for this morphology characteristic may contain a threshold at ten, wherein the values at or above the threshold indicate a high probability of capture. Conversely, a criterion for this morphology characteristic may contain a threshold at five, wherein the values less than or equal to five indicate a high probability of capture.

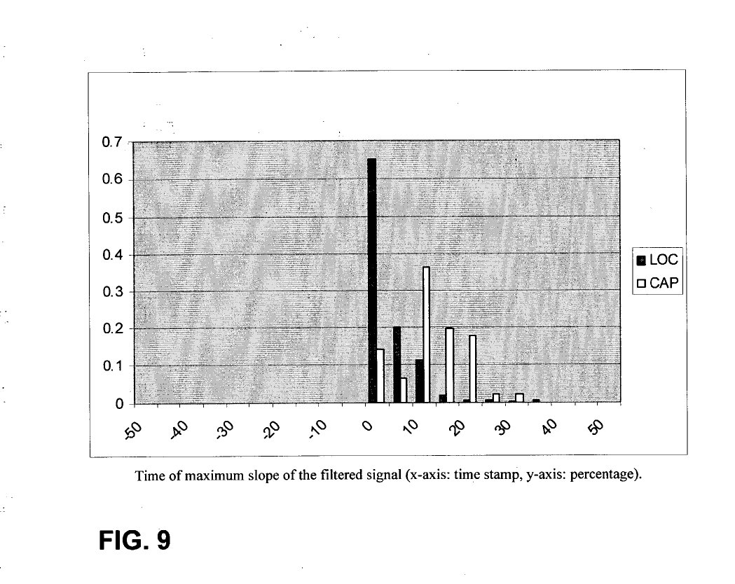

FIG. 9 is a histogram illustrating a time of maximum slope of a filtered signal sensed within a heart chamber following delivery of a pacing pulse. The time characteristic is another morphology characteristic, and refers to the time within the sampling window at which the filtered signal exhibits its maximum slope. The units of the digital values, as seen on the x-axis, are “time stamps.” In the example of FIG. 9, the sample time is 1.26 ms. A time stamp is a specific sample-number within a predefined sampling window. The y-axis describes the occurrence-frequency, with 1 being equal to 100% occurrence rate.

When the time of maximum slope morphology characteristic is less than or equal to approximately five, the graph of FIG. 9 shows the probability of a loss of capture is very high. Therefore, an example criterion for this morphology characteristic may contain a threshold at five, wherein the values above the threshold indicate a high probability of capture. Conversely, a criterion for this morphology characteristic may contain a threshold at five, wherein the values less than or equal to five indicate a high probability of loss of capture.

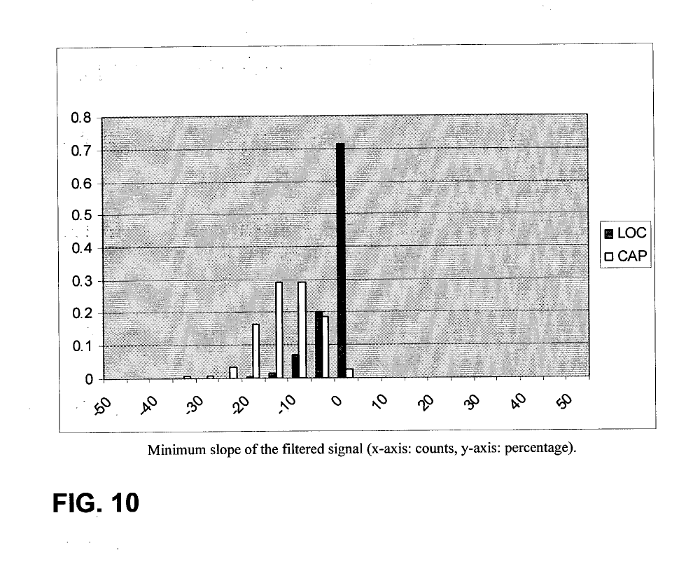

FIG. 10 is a histogram illustrating a minimum slope of a filtered signal sensed within a heart chamber following delivery of a pacing pulse. The minimum slope characteristic represents the minimum slope of the sensed signal waveform within the sampling window. The units of the digital values, as seen on the x-axis, are “counts.” The absolute range of the x-axis in the example of FIG. 9 is ±100 mV. The y-axis describes the occurrence-frequency, with 1 being equal to 100% occurrence rate.

When the minimum slope morphology characteristic is greater than or equal to approximately negative five, the graph shows the probability of a loss of capture is very high. Therefore, an example criterion for this morphology characteristic may contain a threshold at negative five, wherein the values below the threshold indicate a high probability of capture. Conversely, a criterion for this morphology characteristic may contain a threshold at zero, wherein the values at zero indicate a high probability of loss of capture.

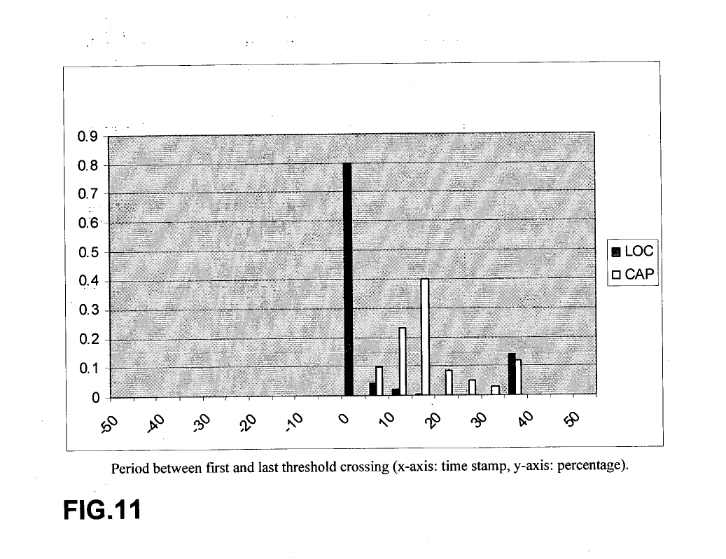

FIG. 11 is a histogram illustrating a period between first and last threshold crossing for a signal sensed within a heart chamber following delivery of a pacing pulse. The units of the digital values, as seen on the x-axis, are “counts.” In the example of FIG. 11, the sample time is 1.26 ms. The y-axis describes the occurrence-frequency, with 1 being equal to 100% occurrence rate.

When the signal width morphology characteristic is less than approximately ten, the graph shows the probability of a loss of capture is very high. When the signal width exceeds ten, however, the likelihood of capture increases. Therefore, an example criterion for this morphology characteristic may contain a threshold at approximately ten, wherein the values at or below the threshold indicate a high probability of loss of capture. Conversely, a criterion for this morphology characteristic may contain a threshold at ten, wherein the values greater than ten indicate a high probability of capture. Note, however, that as the count value approaches 35 to 40, loss of capture becomes as likely as capture. Therefore, the morphology criteria may define a range, rather than merely a threshold. In this example, if the sensed signal width is in the range of approximately ten to thirty, IMD 10 detects CAP. Otherwise, IMD 10 determines either LOC or an indeterminate capture status.

The preceding specific embodiments are illustrative of the practice of the invention. Various modifications may be made without departing from the scope of the claims. For example, the invention may be practiced by a variety of implantable medical devices that perform capture tests. Moreover, the morphology characteristics such as signal width and minimum voltage are exemplary, and the invention is not limited to those characteristics. In addition, the evoked response detection as described herein may be used not only during threshold testing, but also during regular pacing therapy where the aim is to continuously assure capture.

The invention may be embodied as a computer-readable medium that includes instructions for causing a programmable processor to carry out the methods described above. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

These and other embodiments are within the scope of the following claims.

1. A method comprising:
delivering a pacing pulse to a chamber of the heart;
sensing a signal within the chamber following the delivery of the pacing pulse; and
determining whether the pacing pulse captured the chamber of the heart based on one or more morphological characteristics of the sensed signal.

2. The method of claim 1, wherein determining whether the pacing pulse captured the chamber comprises:
processing the sensed signal to identify the morphology characteristic;
comparing the identified morphology characteristic to one or more morphology criteria; and
determining that the pacing pulse captured the chamber when the morphology characteristics satisfy the morphology criteria.

3. The method of claim 2, further comprising determining that the pacing pulse did not capture the chamber if the morphology characteristics do not satisfy the morphology criteria.

4. The method of claim 2, wherein the morphology characteristics include a minimum voltage of the sensed signal.

5. The method of claim 4, wherein the morphology criteria specify a minimum voltage range indicative of capture, the method further comprising determining that the pacing pulse captured the chamber based on comparison of the minimum voltage to the minimum voltage range.

6. The method of claim 2, wherein the morphology characteristics include a minimum slope of the sensed signal.

7. The method of claim 6, wherein the morphology criteria specify a minimum slope range indicative of capture, the method further comprising determining that the pacing pulse captured the chamber based on comparison of the minimum slope to the minimum slope range.

8. The method of claim 2, wherein the morphology characteristics include a width of the signal.

9. The method of claim 8, wherein the morphology criteria specify a width range indicative of capture, the method further comprising determining that the pacing pulse captured the chamber based on comparison of the width to the width range.

10. The method of claim 2, wherein the morphology characteristics include a maximum slope of the sensed signal.

11. The method of claim 10, wherein the morphology criteria specify a maximum slope range indicative of capture, the method further comprising determining that the pacing pulse captured the chamber based on comparison of the maximum slope to the maximum slope range.

12. The method of claim 2, wherein the morphology characteristics include a time of maximum slope of the sensed signal.

13. The method of claim 12, wherein the morphology criteria specify a time of maximum slope range indicative of capture, the method further comprising determining that the pacing pulse captured the chamber based on comparison of the time of maximum slope to the maximum slope range.

14. The method of claim 2, wherein the morphology characteristics include a minimum slope of sensed signal.

15. The method of claim 14, wherein the morphology criteria specify a minimum slope range indicative of capture, the method further comprising determining that the pacing pulse captured the chamber based on comparison of the minimum slope to the minimum slope range.

16. The method of claim 2, wherein the morphology characteristics include a minimum voltage of the sensed signal, a minimum slope of the sensed signal, and a width of the signal.

17. The method of claim 16, further comprising determining whether each of the morphology characteristics satisfies an applicable criterion within the morphology criteria.

18. The method of claim 16, wherein the minimum voltage is a rough sensed minimum voltage.

19. The method of claim 16, wherein the minimum voltage is a filtered minimum voltage.

20. The method of claim 1, further comprising filtering the sensed signal, amplifying the sensed signal, and converting the signal to a digital signal.

21. An implantable medical device comprising:
a sensor to sense a signal from within a chamber of a heart following delivery of a pacing pulse; and
a processor to determine whether the pacing pulse captured the chamber of the heart based on one or more morphological characteristics of the sensed signal.

22. The device of claim 21, wherein the processor is further configured to measure the morphology characteristic.

23. The device of claim 21, further comprising a lead having a proximal end and a distal end, the lead comprising an electrode on the distal end.

24. The device of claim 21, further comprising a pulse generator to generate a pacing pulse for delivery to a chamber of the heart via the electrode.

25. The device of claim 21, wherein the processor is configured to:
process the sensed signal to identify the morphology characteristic;
compare the identified morphology characteristic to one or more morphology criteria; and
determine that the pacing pulse captured the chamber when the morphology characteristics satisfy the morphology criteria.

26. The device of claim 25, wherein the processor determines that the pacing pulse did not capture the chamber if the morphology characteristics do not satisfy the morphology criteria.

27. The device of claim 25, wherein the morphology characteristics include a minimum voltage of the sensed signal.

28. The device of claim 27, wherein the morphology criteria specify a minimum voltage range indicative of capture, the processor determining that the pacing pulse captured the chamber based on comparison of the minimum voltage to the minimum voltage range.

29. The device of claim 25, wherein the morphology characteristics include a minimum slope of the sensed signal.

30. The device of claim 29, wherein the morphology criteria specify a minimum slope range indicative of capture, the processor determining that the pacing pulse captured the chamber based on comparison of the minimum slope to the minimum slope range.

31. The device of claim 25, wherein the morphology characteristics include a width of the signal.

32. The device of claim 31, wherein the morphology criteria specify a width range indicative of capture, the processor determining that the pacing pulse captured the chamber based on comparison of the width to the width range.

33. The device of claim 25, wherein the morphology characteristics include a maximum slope of the sensed signal.

34. The device of claim 33, wherein the morphology criteria specify a maximum slope range indicative of capture, the processor determining that the pacing pulse captured the chamber based on comparison of the maximum slope to the maximum slope range.

35. The device of claim 25, wherein the morphology characteristics include a time of maximum slope of the sensed signal.

36. The device of claim 35, wherein the morphology criteria specify a time of maximum slope range indicative of capture, the processor determining that the pacing pulse captured the chamber based on comparison of the time of maximum slope to the maximum slope range.

37. The device of claim 25, wherein the morphology characteristics include a minimum slope of sensed signal.

38. The device of claim 37, wherein the morphology criteria specify a minimum slope range indicative of capture, the processor determining that the pacing pulse captured the chamber based on comparison of the minimum slope to the minimum slope range.

39. The device of claim 25, wherein the morphology characteristics include a minimum voltage of the sensed signal, a minimum slope of the sensed signal, and a width of the signal.

40. The device of claim 39, wherein the processor is configured to determine whether each of the morphology characteristics satisfies an applicable criterion within the morphology criteria.

41. The device of claim 39, wherein the minimum voltage is a rough sensed minimum voltage.

42. The device of claim 39, wherein the minimum voltage is a filtered minimum voltage.

43. The device of claim 21, further comprising a filter to filter the sensed signal, an amplifier to amplify the sensed signal, and an analog-to-digital converter to convert the signal to a digital signal.

44. A computer-readable medium comprising instructions to cause a programmable processor to:
process a signal sensed from a chamber of the heart following delivery of a pacing pulse to the chamber to identify one or more morphological characteristics; and
determine whether a pacing pulse captured a chamber of the heart based on one or more morphological characteristics of the sensed signal.

45. The computer-readable medium of claim 44, further comprising instructions to cause the processor to:
process the sensed signal to identify the morphology characteristic;
compare the identified morphology characteristic to one or more morphology criteria; and
determine that the pacing pulse captured the chamber when the morphology characteristics satisfy the morphology criteria.

46. The computer-readable medium of claim 44, further comprising instructions to cause the processor to determine that the pacing pulse did not capture the chamber if the morphology characteristics do not satisfy the morphology criteria.

47. The computer-readable medium of claim 44, further comprising instructions to cause the processor to determine whether each of the morphology characteristics satisfies an applicable criterion within the morphology criteria.

48. The computer-readable medium of claim 44, wherein the signal morphology characteristics include at least one of: a maximum slope of the sensed signal, a time of maximum slope of the sensed signal, a minimum slope of the sensed signal, a width of the signal, a minimum voltage of the sensed signal, and a minimum slope of the sensed signal.
